(19) United States
(12) Patent Application Publication
(10) Pub. No.: US 2008/0097604 A1
(43) Pub. Date: Apr. 24, 2008

(54) Device For Blocking A Tendon Graft

(76) Inventors: Michael J. Strobel, Straubing (DE); Andreas Weiler, Berlin (DE); Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 11/563,985

(22) Filed: Nov. 28, 2006

(30) Foreign Application Priority Data
Nov. 28, 2005 (DE) 10 2005 057 111.5

(51) Int. Cl. A61F 2/08

(52) U.S. Cl. 623/13.14

(57) ABSTRACT

A device serves for blocking a tendon graft in a drilled hole. An element is connected to said tendon graft which can be expanded radially. The element has a cylindrical body which is divided by an oblique cut into two wedge-shaped bodies initially connected to one another via a predetermined break point.

### BACKGROUND OF THE INVENTION

The invention relates to a device for blocking a tendon graft in a drilled hole in a bone, the drilled hole having a first portion with a first diameter for receiving the tendon graft, said first portion being adjoined by a second portion of smaller diameter through which pulling threads connected to the tendon graft extend as far as the outer face of the bone.

Tendon grafts of this kind are used in particular for replacing the anterior or posterior cruciate ligament of the knee joint in cases of injury. The operating technique generally used is described in detail in Strobel, Michael: Arthroskopische Chirurgie, Springer, 1998, in Kapitel 2, Kniegelenk, pages 438, 459.

For example, in order to anchor a tendon graft in the femur, a drilled hole is formed in the latter, from the direction of the knee joint, said drilled hole having a first portion with a first diameter for receiving the tendon graft. This first portion is adjoined by a second portion of smaller diameter that extends through the femur to the outer face of the latter. Pulling threads, which are knotted to the graft, are pulled through this second portion and are fixed on the outer face by what is called a fixation button.

It is known from U.S. Pat. No. 6,264,694 B1 to provide an element in the form of a ball at the insertion end of the tendon graft, said ball being connected both to the tendon graft and also to the pulling threads.

The external diameter of this ball is such that it can be inserted into the first portion of the drilled hole with the greater diameter, but cannot pass into the second portion of smaller diameter. After the pulling threads have been drawn taut, the ball-shaped element rests on the shoulder at the transition from the first portion of greater diameter of the drilled hole to the second portion of smaller diameter. This ball ensures that, when the pulling threads are pulled, the tendon graft does not move into the second portion of smaller diameter. However, the ball lies on the shoulder only via one contact line. Depending on the embodiment, the ball can be made of an absorbable material.

To secure the tendon graft against being pulled out of the drilled hole, an interference screw is additionally inserted which additionally presses the ball against the shoulder at the end of the first portion of the drilled hole. It is only when this interference screw is screwed in that the tendon graft is blocked in the first portion of the drilled hole.

A disadvantage of this design is that two structural elements are needed, that is to say the ball and the interference screw, in order to block the tendon graft. Since the longitudinal axis of the interference screw extends in the longitudinal axis of the drilled hole and thus also in the longitudinal extent of the tendon graft received in this portion, the outer face of the interference screw is in direct contact with the tendon graft over a relatively long area. As the interference screw is being screwed in, it is not possible to rule out the possibility of the tendon graft being damaged by the external thread, which can lead to tearing of the tendon graft when it is subjected to loading.

It is an object of the present invention to make available a device for blocking a tendon graft which does not itself damage the tendon graft.

### SUMMARY OF THE INVENTION

According to the invention this object is achieved by the fact that the element is designed to be radially expandable, wherein the radial expandability can be achieved by pulling on at least one draw-thread, said element is approximately cylindrical and is divided by an oblique cut into two wedge-shaped bodies which can be displaced relative to one another in an axial direction, said two wedge-shaped bodies are connected to one another via a predetermined break point.

The blocking is now achieved by the element itself, specifically by its being designed so as to be radially expandable. The element is connected to the insertion end of the tendon graft and can additionally be connected to the pulling threads, and/or it can be provided with separate draw-threads which are connected only to the element and which serve exclusively for the expansion.

The tendon graft and the radially expandable element and its draw-threads are guided into the drilled hole, the draw-threads being guided through the portion of smaller diameter and reaching the outside. By pulling on the draw-threads, the element is guided as far as the shoulder between the first portion of greater diameter and the second portion of smaller diameter. By further pulling on the draw-threads, the element is now radially expanded and in this way is blocked in the drilled hole. The expanded element now sits captive in the drilled hole and, since the tendon graft is connected to the element, the tendon graft too is blocked in the drilled hole.

The radial expandability can be such that, when the element has been expanded, it remains expanded permanently, or it is also possible to have a reversible expandability, i.e. when the tensioning or the tensile force on the draw-threads is removed, the element can radially contract again and the blocking action is cancelled.

This latter property may be desirable, for example, in cases where, once the draw-threads have been tensioned, another position is needed or another tendon graft is to be inserted because the tendon graft has been found to be incorrectly aligned or because defects have been found. It is desirable for the tendon graft to become incorporated into the bone and thus become permanently fixed. In this case, provision can also be made for the element to be made of bioabsorbable material.

The two wedge-shaped bodies have the advantage that, by means of the displacement of the wedges in the axial direction, which is effected simply by pulling on the draw-threads, the radial expansion of the body is achieved, and the extent of the expansion can be selected by the extent of the mutual movement of the wedges. These wedge-shaped bodies can provide particularly effective blocking.

The cylindrical body has the considerable advantage that the geometry of the element corresponds to the geometry of a drilled hole, which is of course cylindrical. Such a cylindrical body can bear across a very large surface area on the shoulder between the transition from the first portion of greater diameter to the second portion of smaller diameter. The outer face of the two wedge-shaped bodies is cylindrical, so that, during the radial expansion caused by displacement of the two wedge-shaped bodies, these come to lie across a large surface area on the inside wall of the drilled hole. This permits particularly effective blocking across a large surface area.

The connection of the two wedge-shaped bodies via a predetermined break point has the advantage that the body is present as one unit prior to the expansion. This considerably facilitates handling, for example during knotting with the draw-threads and upon application to the tendon graft and during the subsequent insertion into the drilled hole. By pulling on the draw-threads, one wedge is thus displaced relative to the other one, resulting in the radial expandability. This pulling force is sufficient to break the predetermined break point, thereby permitting the displaceability of the wedge-shaped bodies.

In another embodiment, at least one catch arrangement is provided on those wedge surfaces of the wedge-shaped bodies that slide along one another.

The catch arrangement is such that the wedge surfaces are able to slide in one direction, in which the wedges are radially expanded, while a reverse sliding is prohibited.

This permits a particularly effective and irreversible blocking of the wedges.

This embodiment allows the operating surgeon initially to bring the assembly of tendon graft, wedge bodies and draw-threads into place, to check the fit, and then, by pulling on the draw-threads, to displace the wedges relative to one another, which displacement can be effected in steps by means of several locking teeth, so that, by gradually firmer pulling, a correspondingly firm blocking can be achieved. The catch arrangement ensures that this procedure is irreversible.

In another embodiment of the invention, the body has, on its outer face, claws which engage with the inner wall of the first portion of the drilled hole when the element is in the expanded state.

This measure has the advantage that, when the body expands, the claws on the outer face eat their way into or catch on the wall of the drilled hole, so that it is possible to achieve a blocking action that is particularly effective against dislodgement of the tendon graft.

In another embodiment of the invention, the element comprises a continuous bore through which the draw-threads can be guided.

This measure has the advantage that the draw-threads can be guided through the bore of the element, so that the outside face of the element is fully available for the blocking effect, that is to say for engagement with the wall of the drilled hole.

In another embodiment of the invention, the draw-threads for the radial expansion of the element are formed by the pulling threads of the tendon graft.

This measure has the advantage that the pulling threads that are present anyway on the tendon graft are at the same time used as draw-threads for expansion of the element. As has already been mentioned, if there is a central continuous opening in the element, these can first be threaded through the latter, so that the tendon graft then bears on one side of the element. By pulling on the pulling threads, the expansion is then effected.

As has already been mentioned, separate draw-threads can be provided or arranged on the element and serve exclusively for expanding the element.

In another embodiment of the invention, the radially expandable element is made of metal, in particular of titanium.

This measure has the advantage of providing a mechanically stable element that can remain permanently in the expanded state in the body.

In another embodiment of the invention, the element is made of absorbable material, in particular of an absorbable polymer.

This measure has the advantage that the tendon graft can gradually become incorporated into the bone, and the body of the expandable element is gradually replaced by regrowth of bone substance.

It will be appreciated that the aforementioned features and those still to be explained below can be used not only in the respectively cited combination, but also in other combinations or singly, without departing from the scope of the present invention.

### BRIEF DESCRIPTION OF THE DRAWINGS

The invention is described and explained in more detail below on the basis of a number of selected illustrative embodiments and with reference to the attached drawings, in which:

FIG. 1 shows a perspective view of a first illustrative embodiment of an element according to the invention for blocking a tendon graft, composed of two wedge-shaped bodies, specifically before expansion,

FIG. 2 shows a perspective view, comparable to FIG. 1, after the radial expansion of the element caused by axial displacement of the wedge-shaped bodies relative to one another,

FIG. 3 shows a plan view of the element from FIG. 1, in the non-expanded state,

FIG. 4 shows a plan view of the element from FIG. 2, in the expanded state,

FIG. 5 shows a partially sectioned view of a device for blocking a tendon graft, with the element depicted in FIG. 1, the assembly made up of tendon graft element and pulling threads having just been inserted into a drilled hole, prior to its radial expansion,

FIG. 6 shows a cross section corresponding to the representation in FIG. 5, after radial expansion of the element in accordance with the change from FIG. 1 to FIG. 2, and

FIG. 7 shows a greatly enlarged cross-sectional representation of a configuration of the wedge surfaces that slide along one another, with a catch arrangement.

### DETAILED DESCRIPTION OF A PREFERRED EMBODIMENT

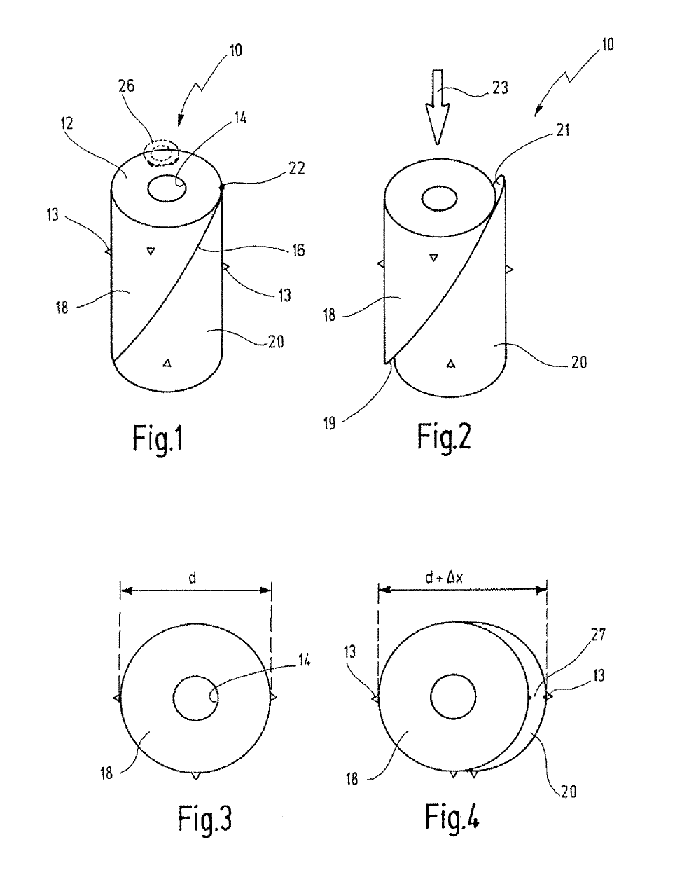

A radially expandable element shown in FIGS. 1 to 4 is designated generally by reference number 10.

The element 10 is composed of a cylindrical body 12 with a continuous bore 14 extending centrally through it.

The body 12 is divided by a diagonal oblique cut 16 into two wedge-shaped bodies 18 and 20. The surface of the cut at the same time defines the two mutually facing wedge surfaces 19 and 21 of the wedge-shaped bodies 18 and 20.

The oblique cut 16 is not entirely continuous, and instead the two wedge-shaped bodies 18 and 20 are still connected to one another via a more or less punctiform predetermined break point 22, so that the body 12 is held in the position shown in FIG. 1, i.e. the two wedge-shaped bodies 18 and 20 do not come apart from one another. On its outside, the body 12 is provided with projections in the form of claws 13 that protrude radially from it. It will be seen from the plan view in FIG. 3 that the body has a diameter d.

When a force is exerted on the wedge-shaped body 18 in the direction of an arrow 23 and when the second wedge-shaped body 20 rests on an abutment, the wedge-shaped body 18 moves in the direction of the arrow 23, after the predetermined break point 22 has been broken.

This force in the direction of the arrow 23 can be exerted by a pulling force, as will be explained in more detail below, for example by a draw-thread connected to the wedge-shaped body 18. The connection can be established via an eyelet 26 provided on the body 18.

In this way, the body 12 is expanded in the radial direction, as can be seen in particular from FIG. 4. This means the radial extent d has increased by the amount Δx, which is dependent on how far the two wedge-shaped bodies 18 and 20 are displaced relative to one another.

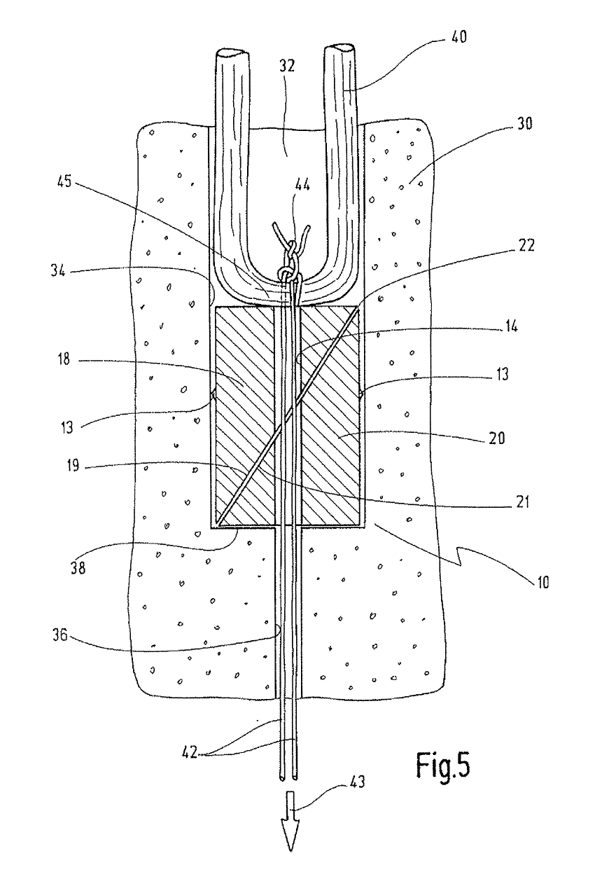

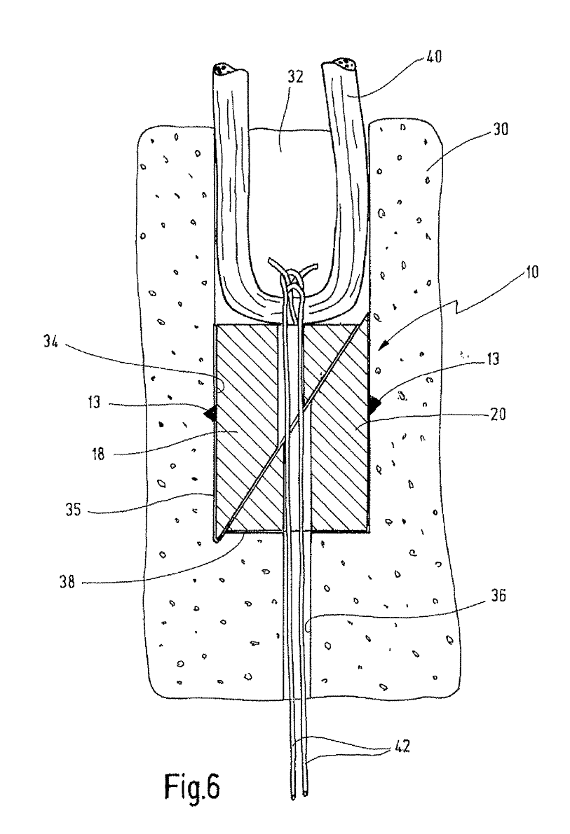

FIGS. 5 and 6 now show how this displacement of the two wedge-shaped bodies 18 and 20 can be achieved.

It will be seen that a drilled hole 32 has been formed in a bone 30, for example in the femur, this drilled hole 32 having a first portion 34 with a first diameter that merges into a second portion 36 with a smaller diameter, the second portion 36 extending as far as the outer face of the femur. A shoulder 38 is formed at the transition between the first portion 34 and the second portion 36.

The clear internal diameter of the first portion 34 corresponds approximately to the external diameter d of the element 10 from FIG. 1, including its claws 13 protruding from the outside face.

A tendon graft 40, which is to be anchored and blocked in the drilled hole 32, is usually formed as a U-shaped loop at its insertion end 45.

The tendon graft 40 can be an artificial graft or, alternatively, a natural tendon that has been taken form another part of the patient's body, for example the semitendinosus tendon.

The insertion end 45 of the tendon graft 40 is knotted with pulling threads 42, and the element 10 is threaded onto the insertion end 45 by the pulling threads 42 being threaded through the central continuous bore 14 in the body 12, and by the element 10 being pushed over the pulling threads 42 as far as the insertion end 45.

If desired, the element 10 can be connected to the tendon graft 40 by an additional knot. In a further embodiment, a separate draw-thread can be provided that is connected only to the upper wedge-shaped body 18, e.g. is knotted to the latter, for which purpose a corresponding eyelet 26 (see FIG. 1) is provided thereon. The expansion of the wedge-shaped bodies 18 and 20 can then be achieved simply via this draw-thread.

This device composed of tendon graft 40, pulling threads 42 and element 10 is now pushed into the drilled hole 32, specifically with the pulling threads 42 toward the front, these pulling threads 42 being threaded through the second portion 36 and reaching as far as the outer face. By pulling on the pulling threads 42, the assembly is pulled into the drilled hole 32 until the leading face of the element 10 comes to lie on the shoulder 38, as is shown in FIG. 5. In this position, the operating surgeon can check for the correct fit.

In order to achieve the blocking effect, the pulling threads 42 are now firmly pulled in the direction of the arrow 43 in FIG. 5. A force is thus exerted on the wedge-shaped body 18. Since the other wedge-shaped body 20 lies on the shoulder 38 as its abutment, the wedge-shaped body 18 slides downward a little along the wedge surfaces 19 and 21, and, as has been described above concerning the change from FIG. 3 to FIG. 4, the element 10 is radially expanded.

This situation after the expansion is shown in FIG. 6. During the relative displacement of the wedge-shaped bodies 18 and 20, the element 10 is blocked in the first portion 34 of the drilled hole 32, and this blocking can be permanently maintained by the pulling threads 42 being secured in this tensioned state. This is done by knotting the pulling threads 42 on the outside onto what is called a fixation button, so that, by subsequently turning this fixation button, it is possible to further adjust this tensioning. The same applies to the embodiment with the separate draw-thread.

During the expansion of the element 10 by means of the relative movement of the wedge-shaped bodies 18 and 20, the claws 13 provided on the outside of the element 10 also eat their way into the inner wall 35 of the drilled hole 32, that is to say into the spongy substance of the bone 30. This provides an additional safeguard against the element 10 being dislodged. The aforementioned tensile force is sufficient to rupture the predetermined break point 22 that connects the two wedge-shaped bodies 18 and 20 to one another, thereby permitting the displacement.

In the design of the element 10 shown in FIGS. 1 to 6, the mutually facing wedge surfaces 19 and 21 are smooth, so that, when the tensioning force on the pulling threads 22 is cancelled, the two wedge-shaped bodies 18 and 20 could in theory be moved back into the position shown in FIG. 5. This design may be desirable if it is found, after insertion and blocking, that the tendon graft is damaged and has to be immediately replaced by another one at the operating site.

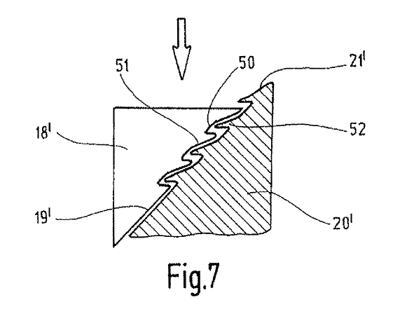

FIG. 7 shows another embodiment in which it will be seen that a catch arrangement 50 is provided on the mutually facing wedge surfaces 19′ and 21′ of two wedge-shaped bodies 18′ and 20′ which in other respects are identical to those described above. The respective teeth 51 and 52 on the mutually facing wedge-shaped bodies 18′ and 20′ are designed in such a way that, as has been described above, a relative displacement is possible during expansion of the body 12, by means of the teeth 51, 52 sliding across one another, whereas this movement is prohibited in the opposite direction.

In this embodiment, the displacement is therefore irreversible, and this results in a secure and irreversible blocking effect.

What is claimed is:

1. A device for blocking a tendon graft in a drilled hole in a bone, said drilled hole having a first portion with a first diameter for receiving said tendon graft, said first portion being adjoined by a second portion of smaller diameter through which pulling threads connected to said tendon graft extend as far as an outer surface of said bone, comprising
an element which can be applied to an insertion end of said tendon graft, which element comes to bear on a shoulder between said first portion and said second portion of said drilled hole, wherein
said element is designed to be radially expandable, a radial expandability can be achieved by pulling on at least one draw-thread,
said element has an approximately cylindrical body,
said cylindrical body is divided by an oblique cut into two wedge-shaped bodies,
said two wedge-shaped bodies can be displaced relative to one another in an axial direction of said drilled hole, and wherein
said two wedge-shaped bodies are connected to one another via a pre-determined break point.

2. The device of claim 1, wherein at least one catch arrangement is provided on wedge surfaces of said wedge-shaped bodies that slide along one another.

3. The device of claim 1, wherein said cylindrical body has, on its outer face, claws which engage with an inner wall of said first portion of said drilled hole, when said cylindrical element is in an expanded state.

4. The device of claim 1, wherein said cylindrical body has a continuous bore through which said draw-threads can be guided.

5. The device of claim 1, wherein said at least one draw-thread for said radial expansion of said cylindrical element is formed by said pulling threads of said tendon graft.

6. The device of claim 1, wherein said cylindrical body is made of metal.

7. The device of claim 6, wherein said metal is titanium.

8. The device of claim 1, wherein said cylindrical body is made of an absorbable material.

9. The device of claim 8, wherein said absorbable material is an absorbable polymer.
